(19) United States
(12) Patent Application Publication
(10) Pub. No.: US 2026/0256448 A1
(43) Pub. Date: Sep. 3, 2026

(54) SYSTEMS AND METHODS FOR SURGICAL MARGIN ASSESSMENT USING INTRAOPERATIVE IMAGING

(71) Applicant: Ultrasound AI Inc., Greenwood Village, CO (US)

(72) Inventor: Robert S. Bunn, Greenwood Village, CO (US)

(21) Appl. No.: 19/575,661

(22) Filed: Mar. 23, 2026

Related U.S. Application Data
(63) Continuation-in-part of application No. 19/264,392, filed on Jul. 9, 2025.
Application No. 19/264,392 is a continuation of application No. 18/431,566, filed on Feb. 2, 2024, now Pat. No. 12,369,883.
Application No. 18/431,566 is a continuation-in-part of application No. 17/573,246, filed on Jan. 11, 2022, now Pat. No. 11,969,289.
Application No. 17/573,246 is a division of application No. 17/352,290, filed on Jun. 19, 2021, now Pat. No. 11,266,376.
(60) Provisional application No. 63/790,426, filed on Apr. 17, 2025.
(60) Provisional application No. 63/443,169, filed on Feb. 3, 2023.
(60) Provisional application No. 63/041,360, filed on Jun. 19, 2020.

(51) Int. Cl. A61B 8/08; A61B 8/00; A61B 8/06; G06T 7/00; G06T 7/20; G16H 10/60; G16H 30/20; G16H 30/40; G16H 50/20

(52) U.S. Cl. CPC A61B 8/0866; A61B 8/06; A61B 8/0875; A61B 8/0883; A61B 8/467; A61B 8/488; G06T 7/0016; G06T 7/20; G16H 10/60; G16H 30/20; G16H 30/40; G16H 50/20; G06T 2207/10132; G06T 2207/20081; G06T 2207/30008; G06T 2207/30044; G06T 2207/30048; G06T 2207/30104

(57) ABSTRACT

A computing system assists surgical margin assessment during a surgical procedure by receiving intraoperative imaging data, classifying frames by view category, and filtering or downweighting uninformative frames. Image analysis logic generates one or more margin scores and maps the score or scores to a spatially localized region adjacent a resection boundary using image patches, windows, activation maps, probe-position metadata, or a combination thereof. A candidate margin output is rendered as a localized overlay, contour, or highlighted region. Feedback logic determines a confidence value for the candidate margin output. When confidence is low, the system outputs a reacquisition instruction identifying the localized region and specifying at least one acquisition adjustment for additional imaging. An optional training workflow correlates pathology findings to intraoperative imaging locations, assigns correlation confidence, and uses sufficiently correlated labeled examples to train later surgical-margin-assessment models.

### CROSS-REFERENCE TO RELATED APPLICATIONS

This application claims priority to U.S. Provisional Application Ser. No. 63/790,426 filed Apr. 17, 2025 and also claims priority to and is a continuation-in-part of U.S. Patent Application Ser. No. 19/264,392, filed Jul. 9, 2025, titled "Artificial Intelligence System for Determining Clinical Values Through Medical Imaging," which is a continuation of U.S. Patent Application Ser. No. 18/431,566, filed Feb. 2, 2024, now U.S. Pat. No. 12,369,883, which is a continuation-in-part of U.S. Patent Application Ser. No. 17/573,246, filed Jan. 11, 2022, now U.S. Pat. No. 11,969,289, which is a continuation of U.S. Patent Application Ser. No. 17/352,290, filed Jun. 19, 2021, now U.S. Pat. No. 11,266,376, and which claims the benefit of U.S. Provisional Patent Application No. 63/041,360, filed Jun. 19, 2020. U.S. Patent Application Ser. No. 18/431,566 also claims the benefit of U.S. Provisional Patent Application No. 63/443,169, filed Feb. 3, 2023. Each of the foregoing patent applications and issued patents are incorporated by reference herein in their entirety.

### TECHNICAL FIELD

This patent application relates to medical imaging and computer-assisted surgical guidance. More particularly, it relates to systems and methods that process intraoperative imaging data to assist assessment of a surgical margin during a resection procedure.

### BACKGROUND OF THE INVENTION

Certain baseline concepts disclosed in the foregoing applications incorporated by reference include image acquisition and storage architectures, image classification and filtering, processing of multiple images, formed-image and raw-sensor-data processing, regression and classification outputs, confidence estimation, and confidence-driven guidance. The present application relies on some of the same concepts, but focuses on surgical-margin-specific adaptations, including spatially localized margin output and an optional pathology-correlated training pipeline.

During oncologic resection, a surgeon often must decide in real time whether tissue adjacent a current resection boundary should be left in place, re-imaged, or further resected. Frozen section pathology, visual inspection, and palpation may provide useful information, but these techniques may be slow, limited in spatial coverage, or difficult to apply continuously during a procedure.

Intraoperative imaging devices can provide repeated views of a surgical field or specimen. However, the resulting data may contain frames that are uninformative because of blood, motion, poor contact, shadows, glare, off-axis orientation, or absence of a meaningful tissue interface. A surgical guidance platform that filters such frames, localizes a region adjacent a resection boundary, and directs reacquisition when confidence is insufficient can improve workflow and reduce avoidable uncertainty.

There is also a need for improved training data in this setting. Pathology findings obtained after tissue removal can be more useful when they are correlated back to locations represented in the intraoperative imaging data, thereby producing higher-value labeled examples for later model training and refinement.

### SUMMARY OF THE DISCLOSURE

In one aspect, a computing system receives intraoperative imaging data from an intraoperative imaging device positioned relative to a surgical site. Image acquisition logic classifies individual frames according to view category, filters or downweights frames that are uninformative, and passes retained frames to image analysis logic.

Image analysis logic processes one or more retained frames to generate one or more margin scores and maps the score or scores to a spatially localized region adjacent a resection boundary. In some embodiments, the mapping is performed using image patches or windows associated with the boundary interface. In some embodiments, the mapping is performed using an activation map generated from retained frames. In some embodiments, probe-position metadata, orientation metadata, or both are used to associate the localized region with a location or direction in the surgical field.

In some embodiments, the one or more margin scores can comprise one or more of: a malignancy likelihood score, residual-disease likelihood score, tissue-differential score, anomaly score, classification score, regression score, confidence-associated score, or a multi-parameter score derived from a plurality of tissue characteristics. By way of example, the plurality of tissue characteristics can include one or more of cellularity, vascularity, density, hydration, inflammation, fibrosis, metabolic activity, or another tissue property associated with malignancy or margin status. The score or scores can be generated per pixel, per voxel, per image patch, per window, per segmented region, per frame, across multiple frames, or as a fused output from multiple data sources.

In some embodiments, the spatially localized region can be represented as one or more coordinates, a location indicator, a direction indicator, a bounding box, a contour, a polygon, a segmentation mask, a saliency map, an attention map, an anomaly map, a heatmap, a probability map, an activation map, or another location-associated representation in image space or in another coordinate system associated with the surgical field. The spatially localized region can be derived from direct model output, post-processing of model output, thresholding of a map, merging of adjacent regions satisfying a criterion, or a combination thereof.

Margin output logic generates a candidate margin output from the localized region. The candidate margin output can take the form of a localized overlay, contour, highlighted region, ranked region of concern, margin flag, or other visual or machine-readable output interface associated with a location in the surgical field.

In some embodiments, the candidate margin output comprises a heatmap overlay, saliency-based overlay, attention-based overlay, anomaly overlay, segmentation overlay, coordinate marker, bounding box, contour, highlighted region, ranked region of concern, categorical recommendation, or augmented-reality overlay associated with the spatially localized region. The output can be presented on a monitor, surgical console, microscope display, heads-up display, wearable display, or another user interface visible during the procedure.

Feedback logic determines a confidence value associated with the candidate margin output. When the confidence value is below a threshold, the system generates a reacquisition instruction identifying the localized region and specifying one or more acquisition adjustments. In some embodiments, the reacquisition instruction is selected by a rule-based policy that maps detected artifacts, low-confidence conditions, missing view categories, or inadequate contact conditions to corrective acquisition actions. When the confidence value satisfies the threshold, the system outputs the candidate margin output on a surgical guidance interface for use during the procedure.

In some embodiments, image analysis logic employs one or more tissue-specific or context-aware models, one or more unsupervised or self-supervised anomaly-detection models, a multi-task learning model having a shared backbone and multiple task-specific output heads, or a multi-modal fusion model configured to combine information from multiple imaging modalities and optionally non-imaging data. Different models, parameter sets, or task heads can be applied to different tissue classes, anatomical regions, imaging modalities, acquisition states, or phases of the procedure.

In another aspect, an optional training workflow correlates pathology findings for removed tissue to locations represented in intraoperative imaging data. Orientation information, specimen-face imaging, fiducial information, or registration operations can be used to create labeled examples identifying margin-positive, margin-negative, or uncertain regions. A correlation confidence value can be assigned to each candidate labeled example so that poorly correlated examples may be excluded or given reduced training weight.

In some embodiments, pathology-correlated training data are assembled from one or more data sources including intraoperative imaging systems, clinical imaging archives, hospital picture archiving and communication systems, specimen images, microscopy images, digital pathology images, and combinations thereof. In various embodiments, candidate labeled examples are generated using data having ground-truth confirmation of margin status, including histopathology-confirmed margin findings, and can include associations between macroscopic intraoperative or specimen imaging and microscopic pathology data.

### BRIEF DESCRIPTION OF THE DRAWINGS

FIG. 1 is a block diagram of a surgical margin assessment system according to various embodiments.

FIG. 2 is a flow chart of an intraoperative margin-assessment workflow according to various embodiments.

FIG. 3 is a flow chart showing image acquisition logic that classifies, filters, and weights intraoperative imaging frames and that supports confidence-based reacquisition according to various embodiments.

FIG. 4 is a flow chart of an optional pathology-correlated training pipeline according to various embodiments.

### DETAILED DESCRIPTION OF PREFERRED EMBODIMENTS

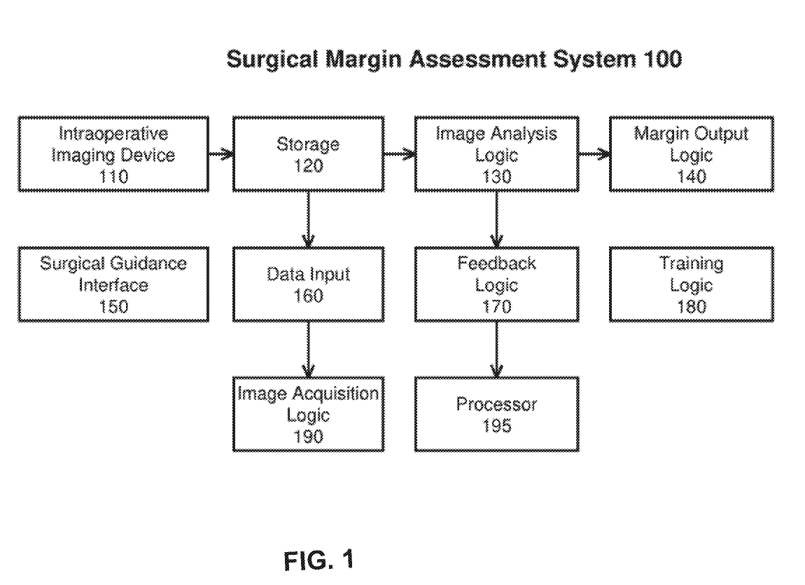

FIG. 1 illustrates a surgical margin assessment system 100. System 100 can include an intraoperative imaging device 110, storage 120, image analysis logic 130, margin output logic 140, a surgical guidance interface 150, optional clinical data input 160, feedback logic 170, training logic 180, image acquisition logic 190, and one or more processors 195. The modules can be implemented on one device or distributed across multiple devices in wired or wireless communication with one another.

Intraoperative imaging device 110 can include an ultrasound device, an optical imaging device, a fluorescence imaging device, an optical coherence device, or another device capable of acquiring imaging data from a surgical field or from tissue removed during a procedure. As described in the incorporated applications, the imaging data can comprise formed images, video frames, or raw sensor data captured before image formation.

Storage 120 stores intraoperative imaging data, model parameters, executable logic, and optional clinical or procedural data. In some embodiments, the stored imaging data includes multiple frames obtained during a sweep or repositioning operation around a surgical site or specimen face. The stored data may represent the same region from multiple angles, depths, or acquisition settings.

Image acquisition logic 190 receives incoming imaging frames and classifies the frames into view categories. Example view categories can include cavity-wall view, tissue-interface view, specimen-face view, normal-tissue view, artifact view, shadowed view, blood-obscured view, and motion-degraded view. Frames classified as uninformative with classification confidence above a threshold can be excluded from downstream analysis or assigned reduced determinative weight. Frames for which the uninformative classification confidence does not satisfy the threshold can be retained for secondary review, additional weighting, or reacquisition analysis.

Image analysis logic 130 processes retained frames and generates one or more margin scores for one or more candidate regions adjacent a resection boundary. The score is indicative of likelihood that a region contains tissue that should be included within a resection margin. The image analysis logic can employ one or more trained neural networks and can use one frame, multiple retained frames, or a combination of retained frames and additional data provided through clinical data input 160.

The one or more margin scores generated by image analysis logic 130 can comprise or include a malignancy likelihood, residual-disease likelihood, tissue-differential score, anomaly score, classification score, regression output, confidence-associated value, or a multi-parameter score based on two or more tissue characteristics associated with tissue at or adjacent a resection boundary. In some embodiments, the two or more tissue characteristics include one or more of cellularity, vascularity, density, hydration, inflammation, fibrosis, or another characteristic correlated with margin status. Image analysis logic 130 can employ one or more trained machine-learning models including, by way of example, convolutional neural networks, transformer models, segmentation networks, generative models, autoencoders, one-class models, anomaly-detection models, tree-based models, or hybrid combinations thereof.

In one implementation, each retained frame or boundary-associated region of interest is divided into overlapping image patches or windows. The system assigns a margin score to respective patches or windows and merges adjacent patches or windows satisfying a criterion to determine a spatially localized region. In another implementation, the system generates an activation map from image features derived from a retained frame or retained frame set and thresholds the activation map to determine a spatially localized region. When probe-position metadata, orientation metadata, depth metadata, or both are available, the localized region can additionally be associated with a direction or location indicator for use in reacquisition guidance.

2In some embodiments, localization is performed using one or more of saliency mapping, attribution analysis, attention mechanisms, anomaly mapping, segmentation output, thresholded probability mapping, coordinate extraction, or combinations thereof. The localized region can be represented as coordinates, a direction indicator, a bounding box, contour, polygon, segmentation mask, saliency map, attention map, anomaly map, heatmap, probability map, activation map, or another image-space or field-registered representation. Where probe-position metadata, orientation metadata, depth metadata, or other position information is available, the representation can be registered to a coordinate system associated with the surgical field or specimen

Margin output logic 140 generates a candidate margin output from the score or scores produced by image analysis logic 130. In some embodiments, the candidate margin output is rendered as a localized overlay superimposed on a displayed image. In some embodiments, the candidate margin output is a contour, highlighted region, ranked list of regions of concern, categorical recommendation, or other output associated with a location represented in the imaging data.

In some embodiments, candidate margin output generated by margin output logic 140 is presented as a heatmap overlay, segmentation overlay, saliency-based overlay, attention-based overlay, anomaly overlay, coordinate marker, bounding box, contour, highlighted region, ranked display of regions of concern, or augmented-reality overlay. Where multiple tissue characteristics are analyzed, the interface can present separate maps for respective characteristics, a combined map derived from multiple characteristics, or a selectable layered display that permits a user to view different score types or localization representations.

Surgical guidance interface 150 presents the candidate margin output to a user. The interface can display the output on a monitor, surgical console, or other display visible during the procedure. The interface can further present a confidence value, acquisition prompt, or status indication showing whether additional imaging is recommended before acting on the candidate margin output.

Feedback logic 170 evaluates the confidence value associated with the candidate margin output. In some embodiments, feedback logic 170 applies a reacquisition policy that maps detected conditions to corrective acquisition actions. By way of example, when image acquisition logic 190 identifies a shadowed or off-axis view with confidence above a threshold, feedback logic 170 can request acquisition at a different angle; when the localized region lies near an edge of the image, feedback logic 170 can request a centered reacquisition or a different depth; when retained frames underrepresent a desired informative view category, feedback logic 170 can request additional frames of that category; and when poor contact is detected, feedback logic 170 can request improved probe contact. The reacquisition policy can be implemented as a rule-based policy, lookup table, or other executable control logic.

When the confidence value is below a threshold, feedback logic 170 directs acquisition of additional imaging of the localized region of concern. The guidance can be output as a user-facing prompt, can be transmitted as a control signal to an imaging device that supports adjustment of acquisition parameters, or can be output in both forms. When the confidence value satisfies the threshold, the candidate margin output can be displayed as a guidance result for the current stage of the procedure.

In various embodiments, training logic 180 assembles candidate labeled examples from intraoperative imaging data, specimen-associated imaging, pathology findings, microscopy or digital pathology data, and optionally additional non-imaging data. In some embodiments, data from multiple institutions, archives, or procedure types are combined to create a more diverse training set. In some embodiments, macroscopic imaging data are correlated with microscopic pathology data so that labels can be generated at different spatial scales for later model training.

Before or during training, candidate labeled examples can be subjected to one or more preprocessing or curation operations including normalization, registration, resampling, artifact correction, segmentation, sanitization, feature selection, feature scaling, augmentation, or combinations thereof. In some embodiments, duplicate examples, outlier examples, poor-quality examples, examples having signal-to-noise ratios below a threshold, or otherwise unreliable examples are removed, filtered, or down-selected before model training.

In various embodiments, training logic 180 uses one or more training strategies including supervised learning using pathologically confirmed labels, self-supervised pretraining followed by fine-tuning, transfer learning, unsupervised learning for anomaly detection or feature discovery, federated learning across multiple sites, multi-modal learning, or combinations thereof. In some embodiments, a neural network is trained using one or more held-out or independent datasets after training and before deployment for later intraoperative use.

In some embodiments, the neural network is configured to receive fused inputs from multiple data sources, including multiple imaging modalities and optionally non-imaging data. Fusion can be performed using early fusion, intermediate fusion, late fusion, attention-based fusion, or other multi-input architectures. By way of example, the fused inputs can include intraoperative imaging, specimen-associated imaging, microscopy or digital pathology, and clinical data associated with a surgical procedure.

Validation can include evaluation on large, diverse, and independent datasets using one or more metrics selected from accuracy, sensitivity, specificity, precision, recall, F1-score, Dice similarity coefficient, intersection over union, area under the receiver operating characteristic curve, mean absolute error, calibration measures, fairness measures, and clinical-utility measures. In some embodiments, validation includes multicenter testing or other testing designed to assess generalizability across patient populations, institutions, imaging systems, or procedure types.

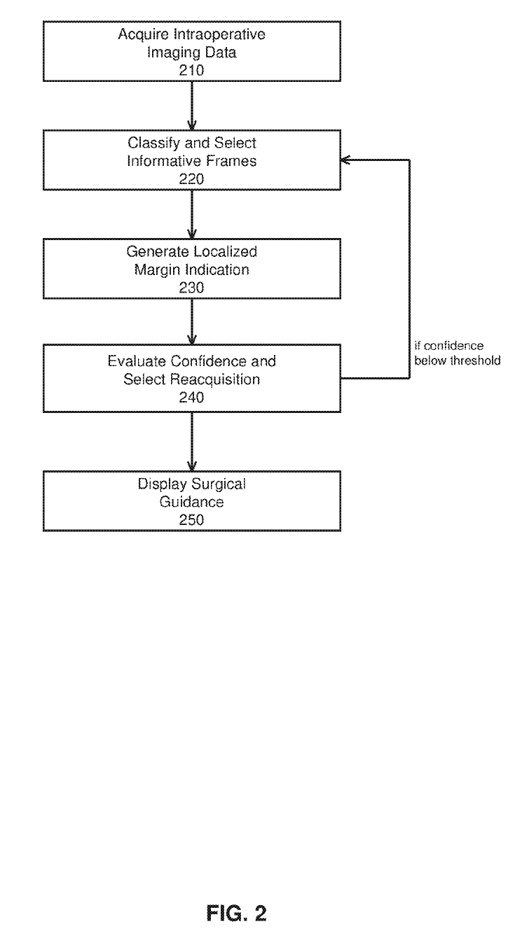

FIG. 2 illustrates one intraoperative workflow. In an acquire imaging step 210, imaging data is acquired from a surgical field. In a classify and filter step 220, image acquisition logic classifies frames by view category and filters or downweights frames classified as uninformative. In a localize candidate margin region step 230, image analysis logic and margin output logic generate a location-associated output for a region adjacent a resection boundary. In a confidence and reacquisition step 240, the system determines whether confidence satisfies a threshold and, if not, selects a reacquisition instruction for additional imaging. If confidence satisfies the threshold, the process proceeds to an output guidance step 250.

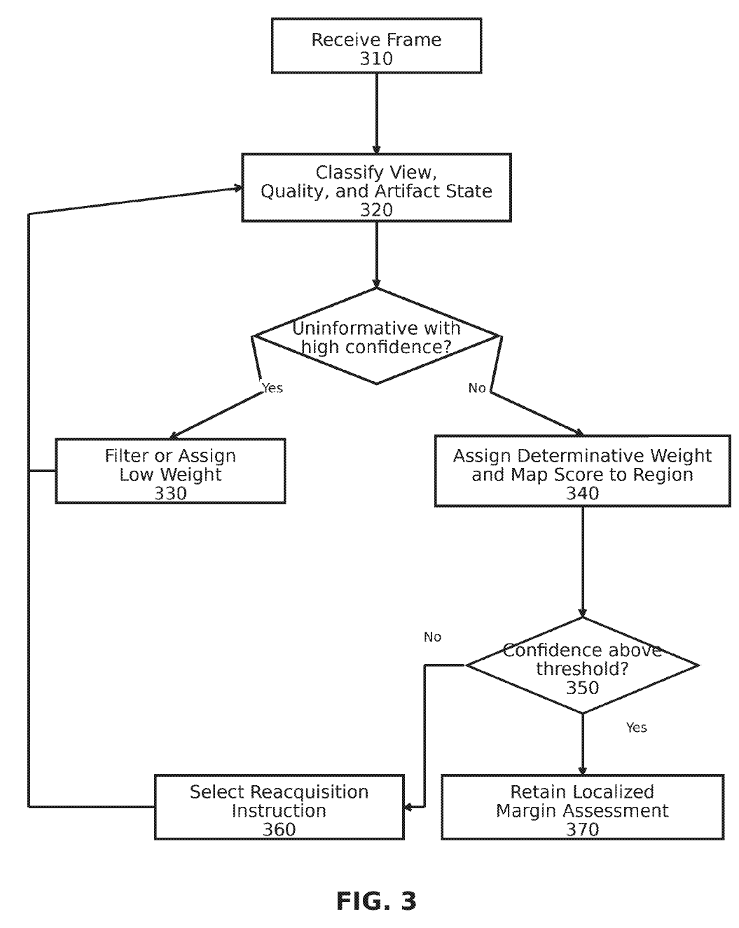

FIG. 3 shows one implementation of image acquisition logic 190. In a receive frame step 310, an incoming frame is received. In a classify view, quality, and artifact-state step 320, the frame is assigned a view category and evaluated for quality. In a filter or assign low weight step 330, frames classified as uninformative with classification confidence above a threshold are excluded from downstream analysis or assigned reduced determinative weight. In an assign determinative weight and map score to region step 340, one or more retained frames are supplied to image analysis logic 130, weighted according to view category, image quality, or both, and mapped to a localized region. In a confidence evaluation step 350, the system determines whether confidence satisfies a threshold. If confidence does not satisfy the threshold, the process proceeds to a select reacquisition instruction step 360 and then returns to receive additional imaging. If confidence satisfies the threshold, the process proceeds to a retain localized margin assessment step 370.

In some embodiments, high-confidence frames are automatically captured and stored for later review or model development. Such stored frames can be used to create curated examples of informative views without requiring a user to manually mark each frame during the procedure. In embodiments using later retraining or dataset refinement, automatically captured frames are not used as training labels unless the frames are independently verified by pathology findings, specimen correlation, qualified reviewer adjudication, or a combination thereof.

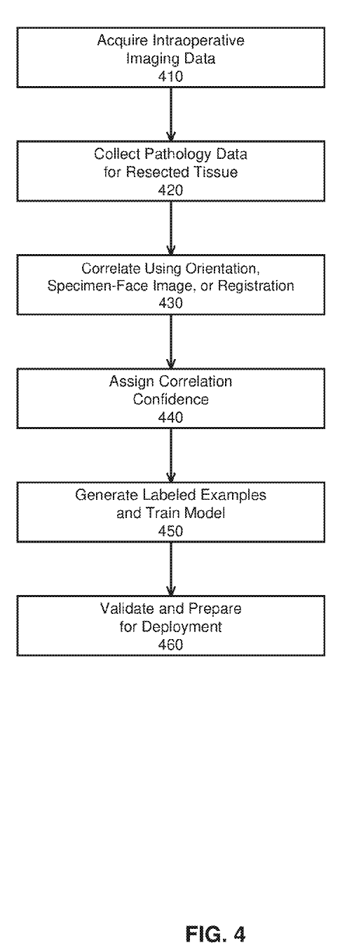

FIG. 4 illustrates an optional pathology-correlated training pipeline 400. In an acquire intraoperative data step 410, intraoperative imaging data is stored from one or more procedures. In a collect specimen information and pathology step 420, removed tissue is associated with orientation information and pathology findings. In a correlate pathology to imaged regions step 430, pathology findings are mapped to regions represented in the intraoperative imaging data. In an assign correlation confidence step 440, a confidence value is assigned to the candidate mapping. In a generate labels and train model step 450, labeled examples are used to train a neural network, and examples with inadequate correlation confidence can be excluded or given reduced weight. In a validate and deploy model step 460, validation is performed on held-out cases and the resulting trained model is made available for later use in intraoperative margin assessment.

Pathology correlation can be carried out using specimen orientation markers, ink markings, sutures, specimen-face images, recorded orientation notes, fiducial landmarks, or registration operations. In some embodiments, pathology findings identify margin-positive regions, margin-negative regions, or uncertain regions. Examples with inadequate correlation quality can be excluded or given reduced weight during training.

The training pipeline can use one or more of the training techniques described in the incorporated applications as referenced above, including use of multiple images, image filtering, processing of raw sensor data, regression outputs, classification outputs, confidence estimation, and guidance logic that favors acquisition of more informative images. The present disclosure adds spatially localized surgical-margin output and pathology-correlated labels for intraoperative margin assessment while keeping the overall platform architecture lean.

3In various embodiments, the trained model used for later intraoperative margin assessment can include one or more tissue-specific or context-aware models configured to apply different analysis parameters to different tissue classes, segmented anatomical regions, or imaging contexts. By way of example, the system can segment input imaging data into different tissue types or anatomical regions and apply distinct models, parameter sets, thresholds, or post-processing logic to respective segments.

In various embodiments, the trained model includes an unsupervised or self-supervised anomaly-detection component configured to identify regions that deviate from surrounding tissue or from expected tissue appearance without requiring explicit labels for every differential. In various embodiments, the trained model includes a multi-task learning architecture in which a shared backbone generates feature representations used by multiple task-specific heads to estimate one or more of: margin likelihood, tissue-differential scores, localization output, segmentation output, and confidence values. In various embodiments, the trained model includes a multi-modal fusion architecture configured to combine information from multiple imaging modalities and optionally non-imaging data using early, intermediate, late, attention-based, transformer-based, graph-based, or other fusion strategies.

For embodiments using raw sensor data instead of formed images, the raw sensor data can be processed as one-channel arrays or other sensor-domain representations before or instead of image formation. By way of example, raw ultrasound data can be envelope-detected, log-compressed, or otherwise converted to a sensor-domain representation suitable for processing while preserving location correspondence to the surgical field.

Although specific embodiments are described, the invention is not limited to the particular examples shown. Features described in connection with one embodiment may be combined with features of another embodiment so long as the resulting combination is not inconsistent with the disclosure.

Several embodiments are specifically illustrated and/or described herein. However, it will be appreciated that modifications and variations are covered by the above teachings and within the scope of the appended claims without departing from the spirit and intended scope thereof.

The “logic” discussed herein is explicitly defined to include hardware, firmware or software stored on a non-transient computer readable medium, or any combinations thereof. This logic may be implemented in an electronic and/or digital device to produce a special purpose computing system. Any of the systems discussed herein optionally include a processor, such as a microprocessor, including electronic and/or optical circuits, configured to execute any combination of the logic discussed herein. The methods discussed herein optionally include execution of the logic by said processor.

Computing systems and/or logic referred to herein can comprise an integrated circuit, a microprocessor, a personal computer, a server, a distributed computing system, a communication device, a network device, cloud computing resource, or the like, and various combinations of the same. A computing system or logic may also comprise volatile and/or non-volatile memory such as random-access memory (RAM), dynamic random access memory (DRAM), static random access memory (SRAM), magnetic media, optical media, nano-media, a hard drive, a compact disk, a digital versatile disc (DVD), optical circuits, and/or other devices configured for storing analog or digital information, such as in a database. A computer-readable medium, as used herein, expressly excludes paper. Computer-implemented steps of the methods noted herein can comprise a set of instructions stored on a computer readable medium that when executed cause the computing system to perform the steps. A computing system programmed to perform particular functions pursuant to instructions from program software is a special purpose computing system for performing those particular functions. Data that is manipulated by a special purpose computing system while performing those particular functions is at least electronically saved in buffers of the computing system, physically changing the special purpose computing system from one state to the next with each change to the stored data.

The embodiments discussed herein are intended to be illustrative of various implementations. As these embodiments are described with reference to illustrations, various modifications or adaptations of the methods and/or specific structures described may become apparent to those skilled in the art. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

1. A computer-implemented method for assisting surgical margin assessment during a surgical procedure, the method comprising:
receiving, by a computing system, intraoperative imaging data from an intraoperative imaging device positioned relative to a surgical site;
classifying, by image acquisition logic executing on the computing system, individual frames of the intraoperative imaging data according to view category and filtering or downweighting frames classified as uninformative;
processing, by image analysis logic executing on the computing system, a set of frames retained from the classifying step to generate one or more margin scores;
mapping the one or more margin scores to a spatially localized region adjacent a resection boundary based on at least one of: (i) scores associated with image patches or windows, (ii) an activation map generated from the retained set of frames, or (iii) probe-position metadata associated with the retained set of frames;
generating, from the one or more margin scores, a candidate margin output comprising a localized overlay, contour, or highlighted region identifying the spatially localized region;
determining a confidence value associated with the candidate margin output;
when the confidence value is below a threshold, generating a reacquisition instruction that identifies the spatially localized region and specifies at least one acquisition adjustment selected from a different imaging angle, a different imaging depth, improved contact, or acquisition of an additional view category, and outputting the reacquisition instruction to the intraoperative imaging device or to a user-facing interface to cause acquisition of additional imaging of the spatially localized region; and
when the confidence value satisfies the threshold, rendering the candidate margin output on a surgical guidance interface.

2. The method of claim 1, wherein the view categories include at least one of: a cavity-wall view, a tissue-interface view, a specimen-face view, a normal-tissue view, a blood-obscured view, a shadowed view, and a motion-degraded view.

3. The method of claim 1, wherein frames classified as uninformative with classification confidence above a threshold are filtered or assigned reduced weight, and frames for which the classification confidence does not satisfy the threshold are retained for secondary review, additional weighting, or reacquisition analysis.

4. The method of claim 1, wherein classifying the individual frames further comprises assigning determinative weights to retained frames based on view category, image quality, or both.

5. The method of claim 1, wherein mapping the one or more margin scores to the spatially localized region comprises assigning respective scores to overlapping image patches or windows and merging adjacent patches or windows that satisfy a criterion.

6. The method of claim 1, wherein the reacquisition instruction is selected by a rule-based policy that maps at least one detected condition to a corrective acquisition action, the at least one detected condition comprising an artifact classification, an underrepresented informative view category, an image-edge localization condition, or inadequate contact.

7. The method of claim 1, wherein the reacquisition instruction is output both as a user-facing prompt and as a control signal to an imaging device capable of adjusting at least one acquisition parameter.

8. The method of claim 1, wherein the intraoperative imaging data comprises formed images, raw sensor data acquired before image formation, or both.

9. The method of claim 1, further comprising automatically capturing and storing frames associated with confidence values above a second threshold for later review or training, wherein frames later used as training labels are independently verified by pathology findings, specimen correlation, qualified reviewer adjudication, or a combination thereof.

10. A surgical margin assessment system, comprising:
an intraoperative imaging device configured to acquire imaging data from a surgical site; storage configured to store the imaging data;
one or more processors; and
memory storing instructions that, when executed by the one or more processors, cause the one or more processors to:
classify individual frames of the imaging data according to view category and filter or downweight frames classified as uninformative;
process a remaining set of frames to generate one or more margin scores;
map the one or more margin scores to a spatially localized region adjacent a resection boundary;
generate a candidate margin output identifying the spatially localized region;
determine a confidence value associated with the candidate margin output;
when the confidence value is below a threshold, generate and output a reacquisition instruction identifying the spatially localized region and specifying at least one acquisition adjustment for acquiring additional imaging of the spatially localized region;
and
when the confidence value satisfies the threshold, render the candidate margin output on a surgical guidance interface.

11. The system of claim 10, wherein the instructions further cause the one or more processors to select the reacquisition instruction using a rule-based policy that maps at least one detected artifact, missing view category, image-edge localization condition, or inadequate contact condition to the at least one acquisition adjustment.

12. A computer-implemented method for generating training data for surgical margin assessment, the method comprising:
acquiring intraoperative imaging data from a plurality of surgical procedures; acquiring pathology data for tissue removed during the plurality of surgical procedures;
correlating the pathology data with locations represented in the intraoperative imaging data using specimen orientation information and at least one of specimen-face imaging, fiducial information, or registration operations;
assigning a correlation confidence value to a candidate labeled example; generating labeled examples that identify margin-positive, margin-negative, or uncertain regions;
and
training a neural network using the labeled examples, wherein candidate labeled examples having correlation confidence values below a threshold are excluded or given reduced training weight.

13. The method of claim 12, wherein validation is performed on held-out cases before the neural network is deployed for later use in assisting surgical margin assessment during a surgical procedure.

14. The method of claim 12, wherein automatically captured intraoperative frames are not used as training labels unless independently verified by pathology findings, specimen correlation, qualified reviewer adjudication, or a combination thereof.

15. A computer-implemented method for assisting surgical margin assessment during a surgical procedure, the method comprising:
receiving, by a computing system, intraoperative imaging data representing tissue at or adjacent a resection boundary from at least one intraoperative imaging modality;
processing, by one or more processors of the computing system using at least one trained machine-learning model, the intraoperative imaging data to identify one or more tissue differentials associated with the resection boundary;
generating, based on the processing, at least one score indicative of likelihood of malignancy for each of a plurality of locations represented in the intraoperative imaging data;
localizing, based on the at least one score, a candidate margin region within the intraoperative imaging data, wherein the localizing comprises generating one or more of a saliency map, an attention map, an anomaly map, a segmentation mask, a heatmap, or a probability map corresponding to the candidate margin region; and
outputting, by the computing system, surgical guidance identifying the candidate margin region on a surgical guidance interface for use in assessing a surgical margin during the surgical procedure.
